(19) United States
(12) Patent Application Publication
(10) Pub. No.: US 2008/0125657 A1
(43) Pub. Date: May 29, 2008

(54) Automated contrast agent augmented ultrasound therapy for thrombus treatment

(76) Inventors: James E. Chomas, San Francisco, CA (US); Anming He Cai, San Jose, CA (US); Richard M. Bennett, Half Moon Bay, CA (US); Ismayil M. Guracar, Redwood City, CA (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/528,882

(22) Filed: Sep. 27, 2006

(51) Int. Cl. A61B 8/14

(52) U.S. Cl. 600/458

(57) ABSTRACT

Control of the sonothrombolysis treatment is automated based on feedback from ultrasound. The region to be treated may be tracked to provide ongoing treatment at the desired location. The treatment may be triggered based on detection of sufficient perfusion. The number or intensity of destructive ultrasound pulses may adapt to the number of remaining contrast agents. The treatment may be ceased or modified based on the efficacy.

Acoustic thrombolysis (sonothrombolysis) uses ultrasound and contrast agents (e.g., microbubbles) to clear clots. For example, U.S. Published patent application Ser. No. ______ (Ser. No. 11/286,983, filed Nov. 23, 2005), the disclosure of which is incorporated herein by reference, discloses the use of low mechanical index (MI) monitoring along with optimized high MI treatment pulses. Most clots form small channels of flow. Optimal clot dissolution is achieved by waiting until agents fill a clot and then delivering a contrast agent destruction pulse for treatment. Continuous delivery of high power pulses may be used. By waiting for agents to enter the clot, the destruction of the contrast agents may clear away small amounts of clot material.

### BACKGROUND

The present embodiments relate to contrast agent augmented ultrasound therapy for thrombus treatment.

### BRIEF SUMMARY

By way of introduction, the preferred embodiments described below include methods, instructions and systems for automated contrast agent augmented ultrasound therapy for thrombus treatment. Control of the sonothrombolysis treatment is automated based on feedback from ultrasound. The region to be treated may be tracked to provide ongoing treatment at the desired location. The treatment may be triggered based on detection of sufficient perfusion. The number or intensity of destructive ultrasound pulses may adapt to the number of remaining contrast agents. The treatment may be ceased or modified based on the efficacy.

In a first aspect, a method is provided for automated contrast agent augmented ultrasound therapy for thrombus treatment. Sonothrombolysis is performed. A processor determines an efficacy of treatment as a function of ultrasound information. The performance of the sonothrombolysis is controlled as a function of the efficacy of treatment.

In a second aspect, a computer readable storage medium has stored therein data representing instructions executable by a programmed processor for automated contrast agent augmented ultrasound therapy for thrombus treatment. The storage medium includes instructions for destroying at least some of the contrast agents with a second ultrasound transmission, determining an efficacy of treatment as a function of ultrasound information responsive to a third ultrasound transmission, and controlling the driving, destroying, or both as a function of the efficacy of treatment.

In a third aspect, a method is provided for automated contrast agent augmented ultrasound therapy for thrombus treatment. A sonothrombolysis is performed with ultrasound. The performance of the sonothrombolysis is automatically controlled with a processor. The automatic controlling includes adapting the performance as a function of ultrasound feedback.

In a fourth aspect, a computer readable storage medium has stored therein data representing instructions executable by a programmed processor for automated contrast agent augmented ultrasound therapy for thrombus treatment. The storage medium includes instructions for destroying at least some of the contrast agents with a second ultrasound transmission, automatically controlling the performance of the driving, destroying, or both as a function of feedback.

The present invention is defined by the following claims, and nothing in this section should be taken as a limitation on those claims. Further aspects and advantages of the invention are discussed below in conjunction with the preferred embodiments and may be later claimed in independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of a system for automated contrast agent augmented ultrasound therapy for thrombus treatment; and

FIG. 2 is a flow chart diagram of one embodiment of a method for contrast agent augmented ultrasound therapy for thrombus treatment.

### DETAILED DESCRIPTION OF THE DRAWINGS AND PRESENTLY PREFERRED EMBODIMENTS

Microbubbles or contrast agents tend to stay in the center of a channel due to the lowest shear condition in the center of the channel. After pushing the contrast agents nearer the channel walls, the contrast agents are destroyed. The destruction from the sonothrombolysis creates flow in regions where there was previously no flow. Since ultrasound can directly measure flow in real-time, the flow information during treatment can be used as feedback to the clinician, the ultrasound system, or the contrast agent injector pump for automating treatment. Rather than providing feedback based only on contrast agent signals, the feedback may be based on the treatment efficacy. The outcome of any sonothrombolysis treatment study is the recanalization of the vessel. Ultrasound is a proven method in imaging Doppler flow. The system can assess treatment performance based on detected Doppler measurements to end automatically the treatment.

Other automation of sonothrombolysis may be provided. The workflow of a sonothrombolysis treatment exam may be improved. Treatment using sonothrombolysis may last tens of minutes. Automation may allow a nurse or doctor to monitor without actually having to sit at the machine and press buttons to track and activate treatment. The sonothrombolysis treatment exam is adapted by the system, reducing the button presses and region orienting performed by the clinician while optimally providing treatment.

The ultrasound system responsible for generating the therapeutic ultrasound also generates an image of a thrombus. The same transmitter and transducer are used for generating B-mode, color Doppler, acoustic radiation force impulse imaging (ARFI), or other imaging and for applying acoustic therapy. The transmitter and/or transducer transmit both imaging pulses and therapeutic pulses. For example, a single linear transducer array with element spacing designed for imaging is also used for therapeutic ultrasound. In alternative embodiments, separate transducers and/or systems are provided for imaging and for therapy.

In one embodiment, a standard ultrasound system, such as the Antares™ or Sequoia® System manufactured by Siemens Medical Solutions USA, Inc. Ultrasound Group, is used with little or no modification. The ultrasound system is capable of generating therapeutic pulses for each of the channels or transducer elements. Since contrast agent disruption is relied on for the therapy, acoustic energy within FDA mechanical index and thermal limitations may be used. Using a standard or modified transducer, the system also generates images by transmission and reception of acoustic energy. The imaging pulses and therapeutic pulses are interleaved and provided from the same transducer.

By imaging and applying therapeutic ultrasound with the same transducer, more directed application of therapeutic ultrasound is provided. A field of view is imaged and a region of interest within the field of view is selected for therapeutic ultrasound. For example, a thrombus area is identified by imaging. The availability of contrast agents in or near the thrombus area is also identified by imaging. Therapeutic ultrasound energy is then transmitted to disrupt the contrast agents at the region of interest.

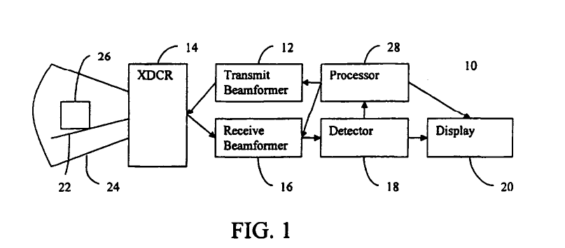

FIG. 1 shows an ultrasound system 10 for contrast agent therapy and imaging using ultrasound energy. The system 10 includes a transmit beamformer 12, a transducer 14, a receive beamformer 16, a processor or detector 18, a display 20, and a processor 28 electrically connected as shown. Additional, different or fewer components may be provided for the system 10. In one embodiment, the system 10 comprises a commercial ultrasound system from one of the manufacturers listed above or another manufacturer.

The transducer 14 comprises a piezoelectric or a capacitive microelectromechanical ultrasound transducer. The transducer 14 has one or more elements for transducing between electrical and acoustical energies. In one embodiment, the transducer 14 includes only a single linear array of elements, such as a flat linear array or a curved linear array. In other embodiments, the transducer comprises a two-dimensional array, a 1.5 dimensional array or other multi-dimensional configurations of elements. The array of elements is configured for insertion into a patient or use external to a patient with or without mechanical rotation or position tracking devices. A mounting may provided for guided, controlled or automated sweeping or movement of the transducer. Alternatively, a wobbler array sweeps. In another alternative embodiment, the transducer is in a catheter, transesophageal, endo-cavity, intra-operative, or other probe for use within a patient.

The transducer 14 is a standard imaging transducer, such as a transducer associated with half wavelength spacing of elements sandwiched between a backing block for absorbing acoustic energy and matching layers for matching the acoustic impedance of the elements to a patient. For example, the transducer is a 4C1 probe available from Siemens Medical Solutions, USA.

In alternative embodiments, the transducer 14 is modified for heat dissipation. For example, a copper foil or copper braid is connected with a lens of the transducer 14 for dissipating heat from the lens. Different piezoelectric materials or matching layers may be optimized for providing a better acoustic or electrical impedance match, reducing an amount of heat generated by the transducer. In one embodiment, multiple layers of piezoelectric or microelectromechanical material separated by electrodes are provided for each element. The multiple layers provide better electrical impedance matching of the transducer to the cable impedance, lowering the generation of heat. In another embodiment, a lensless array or a piezoelectric material shaped to provide elevation focus without a lens focus is provided to reduce the heating of the transducer 14. Reduced heating or more efficient heat dissipation allows for better penetration of acoustic energy and higher power transmissions, such as associated with color Doppler or therapeutic acoustic energy.

The transducer 14 is designed for operation within a frequency band. Typically, the frequency band is associated with transmission and reception of both imaging and therapeutic pulses having a same or similar center frequency. In alternative embodiments, the transducer 14 is associated with wide band operation, such as operating to transmit at a fundamental frequency and receive at a second or third order frequency. The imaging and therapeutic pulses may also be provided at substantially different center frequencies, such as associated with a −6 dB down spectral bandwidth that do not overlap. Any frequency range may be used, but lower ultrasound frequencies (e.g., about or less than 2 MHz center frequency) are used in one embodiment for breaking the contrast agents.

The transmit beamformer 12 is a waveform generator, pulser or other source of electrical excitations for imaging and therapeutic transmissions. In one embodiment, the transmit beamformer 12 generates waveforms for each of a plurality of channels or transducer elements, such as 128 waveforms, and separately delays and apodizes the waveforms for focusing transmissions along scan lines 22 within a field of view 24. Based on the delays and apodization, multiple transmissions may be sequentially scanned across substantially parallel scan lines 22 in the entire field of view 24. The field of view 24 is formed in response to the scan pattern, such as a linear, sector or Vector® scan patterns. Plane wave or diverging wavefronts with or without steering are alternatively formed.

The transmit beamformer 12 electrically connects with the transducer 14 for generating transmissions of acoustic energy or transmit pulses in response to the electrical signals from the transmit beamformer 12. The acoustic energy transmitted includes one of imaging, pushing, or therapy pulses. Imaging pulses are transmissions adapted for generating an image of the field of view 24, such as sequential transmissions of narrow beams sequentially focused along a plurality of scan lines 22.

B-mode, Doppler, and/or other imaging pulses may be used, such as 1-3 cycle B-mode pulses with a mechanical index of 0.1 to 0.4, or up to 1.9. A lower mechanical index (e.g., less than 0.5) may minimize destruction of the contrast agent. Doppler pulses may be the same or different than B-mode pulses. Pushing pulses may be wide or narrow band, such as at least 10 cycles, or 10s to 100s of cycles. Narrowband may more effectively move contrast agents at low powers, such as mechanical index at below 0.1 (e.g., as about 0.01). Therapy pulses include transmissions adapted for contrast agent destruction. Therapy pulses or transmissions are operable to cause rupture of contrast agents. For example, higher power pulses (e.g., about or above 1.2 MI) propagate within a region of interest 26 of the field of view 24. The therapy pulses are focused along scan lines 22 within the region of interest 26. Plane or diverging wavefronts may alternatively be used.

The receive beamformer 16 generates receive beams for imaging. The receive beamformer 16 applies various delays and apodization to electrical signals received from elements of the transducer 14 and sums the signals to generate a receive beam representing a scan line 22 in response to each of the transmissions. The received echoes are responsive to the imaging transmissions. Echoes may or may not be received for imaging in response to the therapy transmissions.

The processor or detector 18 comprises one or more of an application specific integrated circuit, general processor, digital signal processor, other digital circuitry, analog circuitry, a combination thereof or other devices for detecting information from the received, beamformed signals for imaging. In one embodiment, the processor 18 comprises a B-mode and/or Doppler detector. For example, the amplitude of an envelope associated with the received signals is detected. As another example, a frequency shift or velocity, magnitude of a Doppler signal or energy, or variance is detected by Doppler or correlation processing for flow or tissue motion imaging. Single pulse or multiple pulse techniques for contrast agent imaging may be used, such as loss-of-correlation imaging or harmonic imaging using modulation of phase and/or amplitude and subsequent combination of received signals. U.S. Pat. Nos. 6,494,841 and 6,632,177, the disclosures of which are incorporated herein by reference, teach contrast agent imaging techniques. Other contrast agent imaging techniques may be used. Other processors for one-dimensional, two-dimensional or three-dimensional imaging may be used.

A two-dimensional image is generated using any of the B-mode, Doppler and/or contrast agent imaging methods discussed above. The detected information from the processor 18 is provided to the display 20. An image is generated on the display. Various combinations or single types of images are displayed substantially simultaneously, such as one or more of a B-mode, Doppler or contrast agent image. In one embodiment, portions of a field of view 24, such as lateral edges, are shown as B-mode or Doppler images, and another portion, such as a laterally centered portion, is displayed as contrast agent image.

Using the system 10 described above, the field of view 24 is imaged. A suspected thrombus or possible blood clot is identified on the image by the user. In one embodiment, higher power B-mode or color-flow (e.g., Doppler) imaging is used to better identify a stiffening thrombus. Contrast agents are injected. The contrast agents travel to the region of interest 26. The same type of imaging or contrast agent imaging is used to identify when sufficient contrast agents are near or in the thrombus. For example, the same system 10 and transducer 14 transmit low MI (e.g. 0.5 or less) acoustic energy for imaging contrast agents with minimal destruction.

The same system 10, including the same transmitter 12 and transducer 14, is then used to transmit therapeutic pulses. For example, therapeutic transmissions are used to destroy the contrast agents, assisting in breaking the thrombus. In one embodiment, the therapeutic pulses are the same as B-mode or color-flow pulses used for imaging. Alternatively, pulses adapted for maximizing contrast agent destruction are used, such as low frequency acoustic energy with a MI of about but below 1.9. A greater pulse repetition frequency may be used to increase acoustic power applied to the contrast agents.

In alternative embodiments, pushing pulses are transmitted. The pushing pulses may act to move at least some of the contrast agent nearer a thrombus channel wall. Lower mechanical index, longer duration pulses than imaging pulses may more likely move the contrast agents without destruction. The pushing pulses are transmitted after identification of the thrombus, but before at least one destruction pulses. The pushing pulses may or may not be repeated with the repetition of the destruction or therapy pulses.

The processor 28 is the same or different device as the processor or detector 18. The processor 28 is any one or more of the components described above for the detector or processor 18. In one embodiment, the processor 28 is a control processor. The processor 28 automates the sonothrombolysis. Based on input from the detector 18 or other source (e.g., scan converter, filter, or beamformer 16), the processor 28 may adapt the sonothrombolysis based on feedback. For example, identifying the region of interest for treatment is automated based on the image information. As another example, the injection of contrast agent is initiated or varied based on detected contrast agents, efficacy of treatment, and/or tracking of the region of interest. In another example, the transmission initiation, transmission location, and/or number of transmissions for therapy are controlled as a function of image tracking, contrast agent detection, or efficacy of treatment. The capture of relevant images, such as Doppler flow images after each repetition of application of therapy pulses, may occur automatically.

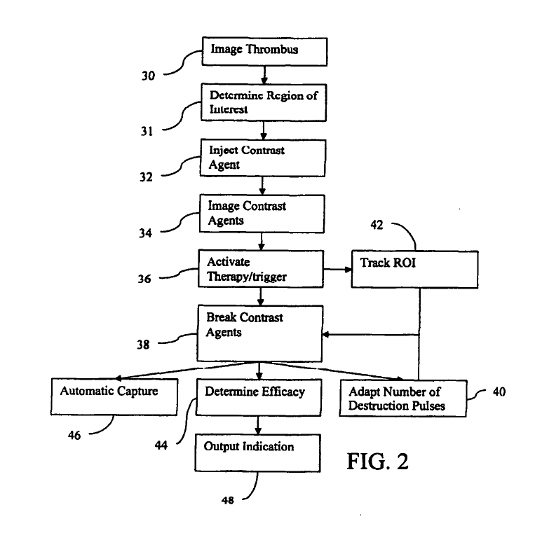

FIG. 2 shows a method of one embodiment for automated contrast agent augmented ultrasound therapy for thrombus treatment. The method is implemented with the system 10 of FIG. 1 or a different system. Additional, different or fewer acts may be performed. For example, the tracking act 42, the determining efficacy act 44, the automatic capture act 46, outputting an indication act 48, the adapting number of pulses act 40, and/or other acts are not provided. The acts are performed in the order shown or a different order. For example, the thrombus is imaged in act 30 after injecting contrast agents in act 32, during the injection of act 32, at a same time as the imaging of contrast agents 34, at other times, or combinations thereof. The imaging acts 30 and 34 may be ongoing while performing other acts, such as acts 36, 38 and 44, or may be discrete events that do not overlap in time with one or more other acts.

One or more of the acts are automated. The performance of the sonothrombolysis is automatically controlled with a processor. For example, select images are captured in act 46, a region of interest is determined in act 31, and/or indications are output in act 48 with little or no user input. The performance may be adaptive as a function of feedback, such as adapting a number of therapy pulses (act 40), adapting continuation of therapy based on efficacy (act 44), and/or adapting therapy transmission location (act 42). The automation may allow sonographers to focus on other matters or require less input or control by the sonographer. For example, the automation may allow for completion of the sonothrombolysis without user input after initiation of the process and/or first therapy pulses.

In act 30, the thrombus or possible blood clot is imaged with the ultrasound transducer. B-mode, color-Doppler and/or another imaging mode allows detection of any thrombosis. Transmitted acoustic energy is high or low MI, such as having an MI greater than 1.0. The frequency used is within the bandwidth of the transducer. In response to the transmissions, echo signals are received using the transducer. The received signals are also responsive to the possible thrombus.

In act 31, using the imaging of act 30, the location of any possible blood clot is identified. Diagnosis of a possible clot may be assisted by applying pressure with the transducer, by the operator, or with another object. The transducer, operator, or other object presses against the patient. A blood clot is less likely than a vein without a blood clot to collapse in response to the external pressure. The difference in flexibility may identify the thrombus.

In one embodiment of act 31, the region of interest is determined for treatment in response to user input and/or automatically. For example, the user chooses or confirms the region of interest. The choice may be in response to processor highlighted or identified tissue markers. Using a classifier or image processing program, regions associated with a thrombus are identified. For example, a correlation of images associated with different external pressures may indicate a location of high correlation along a vessel (e.g., stiffness associated with a thrombus). Any tissue marker may be used, such as the intimal-medial wall in a vessel. Alternatively, the region of interest is determined without user confirmation, such as processor correlation based on user indication of times of different amounts of external pressure.

In act 32, contrast agents are injected. For example, the contrast agents are provided in the blood of a patient through intravenous infusion. Other now known or later developed techniques for introducing contrast agents adjacent to or in the thrombus may be used, such as injection with a needle or through a catheter directly in or near the possible blood clot. The contrast agents may be provided at one time or substantially continuously. For example, an injection pump with variable rates of injection provides the contrast agents over time.

Any contrast agents may be used. In one embodiment, the contrast agents carry drugs or are mixed with drugs, such as drugs for assisting in disruption or weakening of the thrombus (e.g., fibrinolytic agents). In other embodiments, the contrast agents are free of any drugs. The contrast agents may be adapted for disruption, such as by having thinner or thicker walls and/or being more or less elastic.

In act 34, the contrast agents adjacent to or in the thrombus are imaged. For example, the possible blood clot continues to be imaged in act 30. As the contrast agents enter the field of view, the contrast agents are imaged with the same mode of operation in act 34 as act 30. A different mode may be used, such as a contrast agent detection mode of imaging. In another example, the possible clot is imaged with a higher transmit level prior to injection and with a lower transmit level after injection. After the injection of contrast agents occurs and before or after the contrast agents enter the field of view, the same transducer images with low-MI ultrasound. The transmitted acoustic energy is maintained at about 0.5 MI or less. Greater powers may be used. The transducer receives acoustic energy in response to the transmission. The acoustic energy is also responsive to the contrast agents and/or the possible thrombus. Low MI and/or higher frequency imaging generate images with less breaking of the contrast agents than occurs in act 38. Some breakage during imaging may be acceptable. The imaging of contrast agents allows identification of when sufficient contrast agents are near or in the thrombus for treatment.

In one embodiment, after or interleaved with the imaging of act 34, optional pushing pulses are transmitted. Contrast agents at or adjacent to a possible clot are driven or pushed with an ultrasound transmission from an ultrasound transducer. The pushing pulses may act to move contrast agents closer to the clot material to be treated. The pushing pulses may occur automatically, such as in response to detection of contrast agents or sufficient contrast agents, in response to timing, in response to activation of the injection pump, and/or in response to user input.

In act 36, the therapy is activated. The user or the system identifies the location of the possible blood clot. The therapy can be applied to a larger or smaller region than the imaging region and/or region of interest. After sufficient contrast agents are detected at the location, the user activates the therapy. For example, the user presses a button on the transducer. As another example, the user depresses a foot peddle. Other user inputs, such as a button or key on a keyboard or control panel, may be used.

In an alternative embodiment, the system or a processor automatically activates the therapy. Set or predetermined start time and duration are provided for the imaging, pushing pulse, and/or therapy pulses. Alternatively, the therapy is adaptively activated in response to a trigger event, such as perfusion of contrast agent. Perfusion of contrast agents within the treatment area, such as the region of interest, is identified by the processor. Flow characteristics, such as color Doppler signals or spectral values may indicate sufficient perfusion at a gate location or region. Alternatively, intensity or average signal value for the region using contrast agent detection is compared to a threshold. In other embodiments, the change in contrast agent average or other intensity is monitored. When a steady state is reached for a desired time, sufficient perfusion is indicated.

The processor triggers the therapy pulses in response to sufficient perfusion. The higher mechanical index ultrasound pulses operable to destroy at least some contrast agents are transmitted in response to identifying the perfusion.

The triggering of act 36 may be repetitive. For example, sufficient perfusion is subsequently identified again. In response, the therapy pulses are again triggered.

In response to the activation of act 36, mechanical contrast agent destruction therapy is applied in act 38. Sonothrombolysis is performed with ultrasound. The sonothrombolysis may or may not include transmitting pushing pulses. The sonothrombolysis is performed by transmitting acoustic energy to destroy contrast agents. Some or all of the contrast agents in a region of interest are destroyed by ultrasound. Acoustic energy breaks the contrast agents at or adjacent to the possible clot. The disruption caused by the destruction of contrast agents mechanically breaks or weakens the blood clot. Disruption may also or alternatively be caused by expansion or contraction of contrast agents without breaking.

Contrast agents are destroyed or expanded by transmitting high-MI ultrasound, such as acoustic energy with an MI about or above 1.0-1.2 or more. Greater acoustic energy may provide more disruptive destruction of contrast agents, such as transmitting with an MI of about 1.9. The acoustic energy is focused at or near the possible blood clot to provide the greatest destructive power at the possible blood clot. Unfocused or weakly focused acoustic energy may be used.

Contrast agents may more likely be destroyed by pulses at lower frequencies with the same MI. For example, a center frequency of about 2.0 MHz or lower is used. Greater frequencies may be used. The duration of a transmit event for breaking contrast agents is of any length. In one embodiment, the duration is less than 50 microseconds, such as being as short as 10 to 20 microseconds. Short duration may avoid temperatures near thermal limits. Longer durations with the same or lower power may be used. The pulses may be repeated, such as repeating the transmission for a few hundreds of microseconds. Greater, lesser or no repetitions may be used. Different MI and/or thermal limits may be provided for therapy as opposed to imaging.

The transmitted acoustic pulses are square waves, sinusoids or other waveforms with or without an envelope, such as a Gaussian or rectangular envelope. In one embodiment, the pulses have a substantially uniform negative peak pressure. Since the system may not instantaneously generate the desired amplitude, the transmit waveforms are phased to begin with a positive peak pressure. By the second half of the initial cycle of the pulse, the system more likely has ramped to the desired amplitude. The negative peak pressures are more likely uniform, increasing the contrast agent destructive capabilities. In other embodiments, different phasing is provided.

The acoustic energy responsive to the therapy transmissions is not used for imaging. The imaging and breaking transmissions are interleaved, such as providing substantially continuous imaging with more sparse therapy or vice versa. Frame to frame, line-to-line, group of frames, group of lines or other interleaving may be used. Alternatively, the therapy transmissions are also used for imaging. The imaging and the therapy pulses are the same or different.

In act 40, the number of destruction pulses may be automatically controlled. The number of therapy pulses or transmissions adapts to the affect of the pulses on the contrast agents. Where more contrast agents are within the region of interest, such as the clot, act 38 may be repeated to further increase treatment. The processor adaptively applies destruction pulses multiple times to a single line, multiple lines or region. The repetition may be location specific, such as repeating for some locations and not others, or for the entire region of interest.

The adaptation of the number of pulses is based on feedback of contrast agent information. Contrast agents are detected after destroying at least some of the contrast agents. For example, the imaging of act 34 is used after performing act 38 to detect any remaining contrast agents. Any of the detection techniques discussed above for triggering in act 36 may be used. Any threshold amount, such as the same, more, or fewer contrast agents than used for any triggering, may be used. If the contrast agent signal remains high, more destruction pulses are fired. The subsequent therapy pulses may be the same or different than previous pulses, such as altering frequency, mechanical index, focal location, aperture, number of cycles, and/or other characteristic to cause possibly more destruction of contrast agents. The direction of the acoustic wavefront may be altered to more likely position contrast agents into a position for subsequent destruction. The wavefront may be tailored to the vessel morphology and/or flow dynamics, such as transmitting during a low flow portion of the heart cycle.

If the contrast agent signal is low, then the system moves to the next acoustic line or region, and/or proceeds to further imaging. For example, the transmission of therapy pulses ceases until sufficient perfusion of contrast agents is detected again in act 36 with or without imaging pursuant to act 30 prior to perfusion. As another example, pulses are transmitted along different scan lines or at different angles. The acoustic energy is swept through a plane or volume. Mechanical or electrical mechanisms steer or focus the acoustic energy to different locations. Automatic or manual control of the sweep is provided. By scanning an entire blood clot in two or three dimensions, the blood clot is more likely disrupted or weakened. The region for sweeping is the same, larger or smaller than an imaging region.

In act 42, the imaging of acts 30 and/or 34 may be used to track the region of interest. The treatment region is tracked with ultrasound. For example, low mechanical index scanning is used to track based on tissue and/or contrast agent information. Other ultrasound information may be used, such as signals responsive to the therapy transmissions.

The region is tracked in two or three dimensions. By using a transducer capable of three-dimensional scanning (e.g., a multi-dimensional array or a wobbler array), required user movement of the transducer may be avoided to track out-of-plane movement. A one-dimensional array may be used, such as for tracking in two dimensions.

The tracking is performed using speckle tracking, feature tracking, velocity mapping, or other now known or later developed technique. Minimum sum of absolute differences, cross-correlation or other correlation searching may be used to identify a location of the region of interest in subsequent images. Translation and/or rotation are tracked. As the position of the region of interest relative to the transducer changes, the position for therapy and/or the region of interest are updated automatically.

The location of the sonothrombolysis is adapted as a function of the tracking. Transmit beamformer parameters, wobbler parameters, or combinations of both are updated as a function of the tracking. For example, the transmit and/or receive beamforming parameters are updated to maintain the imaging and/or treatment focus within the chosen region of interest. As another example, the wobbling origin and angle sweep in a wobbler transducer are altered to maintain the imaging and/or treatment focus within the chosen region of interest.

If the region of interest significantly decorrelates from the originally defined region of interest, a visual and/or audio alarm may be generated. If the region of interest has moved too much to be accurate, the user may be notified. Further automation is provided by stopping treatment pulses until the user resets the sequence.

In act 44, the efficacy of treatment is determined. A processor determines the efficacy for automated control based on the efficacy. The treatment progress is monitored by any ultrasound imaging mode, such as B-mode two or three-dimensional imaging, color Doppler or spectral Doppler modes. In one embodiment, the ultrasound information used for determining efficacy is responsive to different transmissions than for performance of the sonothrombolysis. For example, pulses with a mechanical index transmission between the low and high mechanical indices of the contrast agent detection pulses and the therapy pulses are used. The imaging of act 30 may be used, such as imaging with power levels for higher resolution or deeper penetration rather than avoiding destruction. The resulting information is used to detect the clot or other indicator of treatment efficacy.

Change in flow, size of the clot, combinations thereof, or other indicator of efficacy may be determined. The treatment may allow for greater volume or velocity of flow. Doppler imaging may detect sufficient or increased flow. Feedback based on detection of flow changes to a vessel or microchannel within a thrombosed vessel indicate efficacy. The treatment may result in a smaller clot size. B-mode and/or Doppler information may indicate sufficient or decreased clot size. Contrast agent or other imaging may indicate differences in the thrombus.

Feedback based on treatment outcome during sonothrombolysis is provided by determining the efficacy. Any threshold may be used to determine sufficient efficacy. The threshold may be predetermined or relative. For example, a percentage change in size, flow, volume flow, or other characteristic identifies completion of the sonothrombolysis.

Different aspects of the sonothrombolysis may be controlled as a function of the efficacy of treatment. The pushing or driving pulses, destruction pulses or imaging pulses may be altered. For example, the location of pushing and/or therapy may be varied. As a portion of a thrombus is sufficiently treated, the focus of subsequent sonothrombolysis may be shifted to insufficiently treated areas. Older clot areas may be more difficult to break up with ultrasound and/or contrast agent destruction. Rather than apply therapy at already removed or broken-up new clot areas, the therapy is applied at the smaller remaining area. The imaging may be shifted to account for the shift of treatment area due to efficacy determination.

In other embodiments, a feed rate of a contrast infusion pump is adjusted as a function of the efficacy of treatment. The adjustment occurs without user input. The rate of infusion may be varied based on the efficacy. If a rate of treatment success is low, the number of contrast agents introduced may be increased. If the treatment is effective, the drip or flow may be reduced or turned off. The adjustment of the infusion rate due to efficacy feedback may minimize contrast agent and/or drug dosage.

The infusion pump may be responsive to other automatic or adaptive control. For example, the rate of infusion may be decreased or stopped where the region of interest shifts. The sonothrombolysis may cease as a function of the shift additionally or alternatively.

The efficacy feedback may indicate substantially complete performance of the sonothrombolysis or inadequate efficacy. In response to one or either, the performance of the sonothrombolysis may be ceased. In addition to or rather than constant monitoring by the user of the sonothrombolysis, the ultrasound system uses efficacy feedback to determine that the process is complete. The ultrasound system ceases the therapy with or without ceasing imaging in response.

In act 48, an indication of the efficacy of treatment is provided for the user. Audio, video and/or another signal indicate completion or other levels of efficacy. For example, a quantitative measure of efficacy, such as flow velocity or vessel volume of blood, is output to the user. Different indications may be used for different user feedback, such as a video indication for level of efficacy and an audio and/or different video feedback indicating completion of the sonothrombolysis. Other feedback may be used. For example, an alarm may sound to notify the clinician if the treatment region of interest has moved significantly and/or treatment has been automatically discontinued. As another example, an audio and/or video flag indicates that the therapy pulses or burst mode of act 38 is active or inactive.

In act 46, images are automatically captured during the sonothrombolysis. Automatic capture of clips throughout the therapy study may assist in diagnosis or verification of efficacy. Images may be captured periodically, such as based on a count or clock. Images may be captures in response to trigger events, such as capturing images used to determine efficacy, images generated during or after application of the therapy pulses, images used to trigger the therapy, and/or other images. Other information may be recorded automatically, such as a quantification of efficacy.

The operations of the system for automated contrast agent augmented ultrasound therapy for thrombus treatment, such as for automated performance of one or more of the acts of FIG. 2 or other acts described herein, or for interaction to provide for manual performance, are implemented with instructions by a programmed processor. The instructions for implementing the processes, methods and/or techniques discussed above are provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A method for automated contrast agent augmented ultrasound therapy for thrombus treatment, the method comprising:
performing sonothrombolysis;
determining, with a processor, an efficacy of treatment as a function of ultrasound information; and
controlling the performance of the sonothrombolysis as a function of the efficacy of treatment.

2. The method of claim 1 wherein performing the sonothrombolysis comprises:
driving contrast agents at or adjacent to a possible clot with a first ultrasound transmission from an ultrasound transducer; and
destroying at least some of the contrast agents with a second ultrasound transmission.

3. The method of claim 2 wherein driving comprises driving with low mechanical index transmission of at least 10 cycles, and destroying comprises destroying with a high mechanical index transmission.

4. The method of claim 2 further comprising:
detecting contrast agents after destroying at least some of the contrast agents; and
adapting a number of second ultrasound transmissions as a function of the detected contrast agents.

5. The method of claim 1 wherein determining the efficacy of treatment comprises detecting with the ultrasound information responsive to different transmissions than for performance of the sonothrombolysis.

6. The method of claim 1 wherein determining the efficacy of treatment comprises detecting change in flow, size of clot, or combinations thereof.

7. The method of claim 1 wherein controlling comprises adjusting, without user input, a feed rate of a contrast infusion pump as a function of the efficacy of treatment.

8. The method of claim 1 wherein controlling comprises ceasing performance for substantially completed sonothrombolysis, a detected change in position, or combinations thereof.

9. The method of claim 1 wherein controlling comprises adapting a location of the sonothrombolysis as a function of region tracking, position of relatively less efficacy of treatment, or combinations thereof.

10. The method of claim 1 further comprising:
generating an indication of the efficacy of treatment for the user.

11. In a computer readable storage medium having stored therein data representing instructions executable by a programmed processor for automated contrast agent augmented ultrasound therapy for thrombus treatment, the storage medium comprising instructions for:
destroying at least some of the contrast agents with a first ultrasound transmission;
determining an efficacy of treatment as a function of ultrasound information responsive to a second ultrasound transmission; and
controlling the destroying as a function of the efficacy of treatment.

12. The instructions of claim 11 further comprising:
driving with low mechanical index transmission of at least 10 cycles;
wherein destroying comprises destroying with a high mechanical index transmission, and determining comprises determining with a mechanical index transmission between the low and high mechanical indices.

13. The method of claim 11 further comprising:
detecting contrast agents after destroying at least some of the contrast agents; and
adapting a number of second ultrasound transmissions as a function of the detected contrast agents.

14. The method of claim 11 wherein determining the efficacy of treatment comprises detecting change in flow, size of clot, or combinations thereof.

15. A method for automated contrast agent augmented ultrasound therapy for thrombus treatment, the method comprising:
performing a sonothrombolysis with ultrasound; and
automatically controlling the performance of the sonothrombolysis with a processor, the automatic controlling comprising adapting the performance as a function of ultrasound feedback.

16. The method of claim 15 wherein performing the sonothrombolysis comprises:
driving contrast agents at or adjacent to a possible clot with a first ultrasound transmission from an ultrasound transducer; and
destroying at least some of the contrast agents with a second ultrasound transmission.

17. The method of claim 15 wherein automatically controlling comprises determining, with the processor, an efficacy of treatment as a function of ultrasound information, and ceasing or altering the performance of the sonothrombolysis as a function of the efficacy of treatment.

18. The method of claim 15 wherein automatically controlling comprises:
identifying, with the processor, perfusion of contrast agents within a treatment area; and
triggering transmission of high mechanical index ultrasound pulses operable to destroy at least some contrast agents in response to identifying the perfusion.

19. The method of claim 15 wherein automatically controlling comprises:
adapting a number of ultrasound transmissions operable to destroy contrast agents, the adapting being as a function of detected contrast agents after transmission of one of the ultrasound transmissions operable to destroy the contrast agents.

20. The method of claim 15 wherein automatically controlling comprises:
tracking a treatment region with ultrasound; and
adapting the location of the sonothrombolysis as a function of the tracking.

21. The method of claim 20 wherein adapting comprises updating transmit beamformer parameters, wobbling parameters, or combinations of both as a function of the tracking.

22. The method of claim 15 further comprising:
automatically capturing images during the sonothrombolysis.

23. In a computer readable storage medium having stored therein data representing instructions executable by a programmed processor for automated contrast agent augmented ultrasound therapy for thrombus treatment, the storage medium comprising instructions for:
driving contrast agents at or adjacent to a possible clot with a first ultrasound transmission from an ultrasound transducer;
destroying at least some of the contrast agents with a second ultrasound transmission;
automatically controlling the performance of the driving, destroying, or both as a function of feedback.

24. The instructions of claim 23 wherein automatically controlling comprises determining an efficacy of treatment as a function of ultrasound information, and ceasing or altering the performance of the sonothrombolysis as a function of the efficacy of treatment.

25. The instructions of claim 23 wherein automatically controlling comprises:
triggering transmission of high mechanical index ultrasound pulses operable to destroy at least some contrast agents;
adapting a number of ultrasound transmissions operable to destroy contrast agents, the adapting being as a function of detected contrast agents after transmission of one of the ultrasound transmissions operable to destroy the contrast agents;
adapting the location of the sonothrombolysis as a function of tracking; or
combinations thereof.
